(19) United States
(12) Patent Application Publication
(10) Pub. No.: US 2024/0312024 A1
(43) Pub. Date: Sep. 19, 2024

(54) METHOD FOR DETECTING AND TRACKING IN A VIDEO STREAM A FACE OF AN INDIVIDUAL WEARING A PAIR OF SPECTACLES

(71) Applicant: FITTINGBOX, LABEGE (FR)

(72) Inventors: Ariel CHOUKROUN, SAINT ORENS DE GAMEVILLE (FR); Jérome GUENARD, TOULOUSE (FR)

(21) Appl. No.: 18/261,233

(22) PCT Filed: Jan. 13, 2022
(86) PCT No.: PCT/FR2022/050067

§ 371 Date: Jul. 12, 2023

(30) Foreign Application Priority Data
Jan. 13, 2021 (FR) 2100297

(51) Int. Cl. G06T 7/20; G06T 5/50; G06T 7/70; G06T 17/00; G06V 10/46

(52) U.S. Cl. CPC G06T 7/20; G06T 5/50; G06T 7/70; G06T 17/00; G06V 10/46; G06T 2207/20221; G06T 2207/30201

(57) ABSTRACT

A method is provided for tracking a face of an individual in a video stream acquired by an image-acquisition device, the face wearing a pair of spectacles. The method includes evaluating parameters of a representation of the face including a model of the pair of spectacles and a model of the face so that the representation of the face is superimposed on the image of the face in the video stream, the parameters being evaluated with respect to a plurality of characteristic points of the representation of the face, previously detected in an image of the video stream, referred to as first image, wherein all or some of the parameters of the representation are evaluated by taking account of at least one proximity constraint between at least one point of the model of the face and at least one point of the model of the pair of spectacles.

### CROSS REFERENCE TO RELATED APPLICATIONS

This application is a National Stage of International Application No. PCT/FR2022/050067 having an International Filing Date of 13 Jan. 2022, which designated the United States of America, and which International Application was published under PCT Article 21(2) as WO Publication No. 2022/153009, which claims priority from and the benefit of French Patent Application No. 2100297, filed on 13 Jan. 2021, the disclosures of which are incorporated herein by reference in their entireties.

### BACKGROUND

### Field

The field of the disclosure is that of image analysis.

More precisely, the disclosure relates to a method for detecting and tracking in a video stream a face of an individual wearing a pair of spectacles.

The disclosure finds in particular applications for the virtual trying on of a pair of spectacles. The disclosure also finds applications in augmented or diminished reality on a face wearing spectacles, with in particular the obscuring at the image of the pair of spectacles worn by the individual, combined or not with the addition of lenses, jewelry and/or makeup. The disclosure also finds applications for taking ophthalmic measurements (PD, monoPD, heights, etc.) on a pair of spectacles worn in reality or virtually by an individual.

### BRIEF DESCRIPTION OF RELATED DEVELOPMENTS

Techniques that make it possible to detect and track a face of an individual in a video stream are known from the prior art.

These techniques are generally based on the detection and tracking of characteristic points of the face, such as a corner of the eyes, a nose or a corner of a mouth. The quality of detection of the face generally depends on the number and position of the characteristic points used.

These techniques are generally reliable for detecting and tracking a face of an individual without an accessory in a video stream.

Such techniques are in particular described in the French patent published under the number FR 2955409 and in the international patent application published under the number WO 2016/135078 of the company filing the present patent application.

However, when the individual is wearing a pair of spectacles comprising corrective lenses, the quality of detection of the face has a tendency to degrade since some of the characteristic points used during the detection, generally the corners of the eyes, are generally deformed by the lenses assembled in the frame, or even masked when the lenses are tinted. Furthermore, even if the lenses are not tinted, it may happen that the frame masks some of the characteristic points used in the detection. When some of the characteristic points are invisible or the position thereof in the image is deformed, the face detected, represented by a model, is generally offset in position and/or in orientation with respect to the real face, or even to the wrong scale.

None of the current systems simultaneously responds to all the requirements, namely proposing a technique for tracking a face wearing a real pair of spectacles, which is more precise and more robust to the movements of the individual, in order to offer an improved augmented-reality rendition.

### SUMMARY

The present disclosure aims to remedy all or some of the above-mentioned drawbacks of the prior art.

For this purpose, the disclosure relates to a method for tracking a face of an individual in a video stream acquired by an image-acquisition device, the face wearing a pair of spectacles, the video stream comprising a plurality of successively acquired images.

The tracking method comprises a step of evaluating parameters of a representation of the face comprising a model of the pair of spectacles and a model of the face so that said representation of the face is superimposed on the image of the face in the video stream.

According to the disclosure, in evaluating all or some of the parameters of the representation, account is taken of at least one proximity constraint between at least one point of the model of the face and at least one point of the model of the pair of spectacles.

By way of example, a proximity constraint may for example define that an arm of the pair of spectacles rests at the junction between the auricle of the ear and the cranium, on the top side, namely at the helix.

In other words, the proximity constraint is defined between a zone of the model of the face and a zone of the model of the pair of spectacles, the zone being able to be a point or a set of points, such as a surface or a ridge.

Proximity means a distance of zero or less than a predetermined threshold, for example of the order of a few millimeters.

Thus the use of a proximity constraint during the evaluation of the parameters of the representation of the face makes it possible to obtain a more faithful pose of the representation of the face with respect to the camera, with a limited number of calculations. A real-time tracking of the individual can consequently be implemented more robustly with regard to unexpected movements of the individual with respect to the image-acquisition device.

Furthermore, the conjoint use of the model of the pair of spectacles and of the model of the face makes it possible to improve the position of the face, in particular compared with the tracking of a face without spectacles. This is because, in the latter case, the position of the characteristic points of the temples is generally imprecise. Tracking the pair of spectacles makes it possible to provide a better estimation of the pose of the representation of the face since the arms of the pair of spectacles superimposed on the temples of the individual make it possible to obtain more precise information on the characteristic points detected in a zone of the image comprising a temple of the individual.

Preferentially, the parameters of the representation comprise values external to the representation of the face and values internal to the representation of the face, the external values comprising a three-dimensional position and a three-dimensional orientation of the representation of the face with respect to the image-acquisition device, the internal values comprising a three-dimensional position and a three-dimensional orientation of the model of the pair of spectacles with respect to the model of the face, said parameters being evaluated with respect to a plurality of characteristic points of said representation of the face, previously detected in an image of the video stream, referred to as first image, or in a set of images acquired simultaneously by a plurality of image-acquisition devices, the set of images comprising said first image.

In other words, the representation of the face, which may be termed avatar, comprises external positioning and orientation parameters in a three-dimensional environment, and relative internal positioning and orientation parameters between the model of the face and the model of the pair of spectacles. Other internal parameters may be added such as the parameters of configuration of the pair of spectacles: type of frame, size of frame, material, etc. The configuration parameters may also comprise parameters related to the deformation of the frame of the pair of spectacles and in particular the arms, when the pair of spectacles is worn on the face of the individual. Such configuration parameters may for example be the angles of opening or closing of the arms with respect to a reference plane such as a principal, or tangent, plane of the face of the pair of spectacles.

The representation of the face comprises three-dimensional models of the face and of the pair of spectacles.

In particular aspects of the disclosure, all or some of the parameters of the representation are updated with respect to the position of all or some of the characteristic points, tracked or detected, in a second image of the video stream or in a second series of images acquired simultaneously by the plurality of image-acquisition devices, the second set of images comprising said second image.

Thus the updating of the parameters of the representation, and in particular of the relative positioning and orientation values between the model of the pair of spectacles and the model of the face, or even of the configuration parameters, makes it possible to obtain a tracking of the face of the individual that is more robust and more precise.

Advantageously, the second image or the second set of images presents a view of the face of the individual at an angle distinct from that of the first image or of the first set of images.

In particular aspects of the disclosure, in evaluating all or some of the parameters of the representation, account is also taken of at least one proximity constraint between a three-dimensional point of one of the models included in the representation of the face and at least one point, or a level line, included in at least one image of the video stream.

In particular aspects of the disclosure, in evaluating all or some of the parameters of the representation, account is also taken of at least one dimension constraint of one of the models included in the representation of the face.

In particular aspects of the disclosure, the method comprises a step of pairing two distinct points belonging either to one of the two models included in the representation of the face, or each to a distinct model from the models included in the representation of the face.

The pairing of two points makes it possible in particular to constrain a distance relationship between these two points, such as a proximity or a known dimension between these two points. A known dimension is for example an interpupillary distance for a face, a width of a frame, a characteristic or mean size of an iris, or any combination of these values according to one or more distribution laws around a known mean value of one of these values.

In particular aspects of the disclosure, the method comprises a prior step of pairing a point of one of the two models included in the representation of the face with at least one point of an image acquired by an image-acquisition device.

The pairing of a point of the model with a point of an image or a set of points such as a contour line is generally implemented automatically.

In particular aspects of the disclosure, during the evaluation of the parameters of the representation, an alignment of the model of the pair of spectacles with an image of the pair of spectacles in the video stream is implemented consecutively with an alignment of the model of the face with an image of the face in the video stream.

In particular aspects of the disclosure, the alignment of the model of the face is implemented by minimizing the distance between characteristic points of the face detected in the image of the face and characteristic points of the model of the face projected in said image.

In particular aspects of the disclosure, the alignment of the model of the pair of spectacles is implemented by minimizing the distance between at least a part of the contour of the pair of spectacles in the image and a similar contour part of the model of the pair of spectacles projected in said image.

It must in fact be emphasized that the model of the pair of spectacles is a 3D model. A projection of this 3D model is thus implemented in the image in order to determine a similar contour that is used in the calculation of minimization of the distance with the contour of the pair of spectacles detected in the image.

In particular aspects of the disclosure, the parameters of the representation also comprise a set of configuration parameters of the model of the face and/or a set of configuration parameters of the model of the pair of spectacles.

The configuration parameters of the model of the face or those of the model of the pair of spectacles can for example be morphological parameters characterizing respectively the shape and the size of the model of the face or those of the model of the pair of spectacles. The configuration parameters can also comprise deformation characters of the model, in particular in the context of the pair of spectacles, to take account of the deformation of an arm or even of the face of the pair of spectacles, or even of the opening/closing of each arm with respect to the front of the pair of spectacles.

In the context of the face model, the configuration parameters can also comprise parameters of opening and closing of the eyelids or of the mouth, or parameters related to the deformations of the surface of the face due to expressions.

In particular aspects of the disclosure, the parameters of the representation comprise all or part of the following list:
a three-dimensional position of the representation of the face;
a three-dimensional orientation of the representation of the face;
a size of the model of the pair of spectacles;
a size of the model of the face;
a relative three-dimensional position between the model of the pair of spectacles and the model of the face;
a relative three-dimensional orientation between the model of the pair of spectacles and the model of the face;
one or more parameter(s) of the configuration of the model of the pair of spectacles;
one or more parameter(s) of the configuration of the model of the face;
one or more parameter(s) of the camera.

In particular aspects of the disclosure, the tracking method comprises steps of:
detection of a plurality of points of the face in a first image of the video stream;
initialization of the set of parameters of the model of the face with respect to the image of the face in said first initial image;
detection of a plurality of points of a pair of spectacles worn by the face of the individual in a second image of the video stream, referred to as second initial image, the second initial image being either subsequent to or prior to the first initial image in the video stream, or identical to the first image in the video stream;
initialization of the set of parameters of the model of the pair of spectacles with respect to the image of the pair of spectacles in said second initial image.

In particular aspects of the disclosure, the initialization of the parameters of the model of the face is implemented by means of a deep learning method analyzing all or some of the detected points of the face.

In particular aspects of the disclosure, the deep learning method also determines the initial position of the model of the face in the three-dimensional reference frame.

In particular aspects of the disclosure, the tracking method also comprises a step of determining a scale of the image of the pair of spectacles worn by the face of the individual by means of a dimension in the image of an element of known size of the pair of spectacles.

In particular aspects of the disclosure, the scale is determined by means of a prior recognition of the pair of spectacles worn by the face of the individual.

In particular aspects of the disclosure, the images acquired by a second image-acquisition device are used to evaluate the parameters of the representation.

In particular aspects of the disclosure, the model of the pair of spectacles of the representation corresponds to a prior modeling of said pair of spectacles, and varies solely in deformation.

The shape and the size of the model of the pair of spectacles remaining invariant, this makes it possible to obtain better resolution in a shorter calculation time.

The disclosure also relates to an augmented reality method comprising steps of:
acquiring at least one stream of images of an individual wearing a pair of spectacles on their face by means of at least one image-acquisition device;
tracking the face of the individual by a tracking method according to any one of the preceding aspects, a position and an orientation of a representation of the face;
modifying of all or some of the images of said or of one of said image streams, referred to as main video stream, acquired by the image-acquisition device or by one of the image-acquisition devices, referred to as main image-acquisition device, by means of the representation of the face superimposed in real time on the face of the individual on the main video stream;
displaying on a screen said previously modified main video stream.

It must be emphasized that the steps of the augmented reality method are advantageously implemented in real time.

The disclosure also relates to an electronic device including a computer memory storing instructions of a tracking or augmented reality method according to any one of the preceding aspects.

Advantageously, the electronic device comprises a processor able to process instructions of said method.

### BRIEF DESCRIPTION OF THE FIGURES

Other advantages, aims and particular features of the present disclosure will appear from the following non-limiting description of at least one particular aspect of the devices and methods that are objects of the present disclosure, with reference to the appended drawings, wherein:

FIG. 1 is a schematic view of an augmented reality device implementing an aspect of the detection and tracking method according to the disclosure;

FIG. 2 is a block diagram of the detection and tracking method implemented by the augmented reality device of FIG. 1;

FIG. 3 shows a view of the mask of a pair of spectacles (sub-figure a) and of the distribution of the points of the contour of the mask according to categories (sub-figures b and c);

FIG. 4 is a perspective view of the face of a model of a pair of spectacles, with and without external envelope (respectively sub-figure b and a);

FIG. 5 illustrates the regression step of the method of FIG. 2, by means of an extract of an image acquired by the image-acquisition device of the device of FIG. 1, on which a model of a pair of spectacles is superimposed;

FIG. 6 illustrates the positioning constraints between a model of the pair of spectacles and a model of the face;

FIG. 7 is a perspective view of a parametric model (3DMM) of a pair of spectacles;

FIG. 8 is a simplified view of the face of the parametric model of FIG. 7.

### DETAILED DESCRIPTION

This description is given on a non-limitative basis, each feature of an aspect advantageously being able to be combined with any other feature of any other aspect advantageously.

It should be noted, as of now, that the figures are not to scale.

### Example of a Particular Aspect

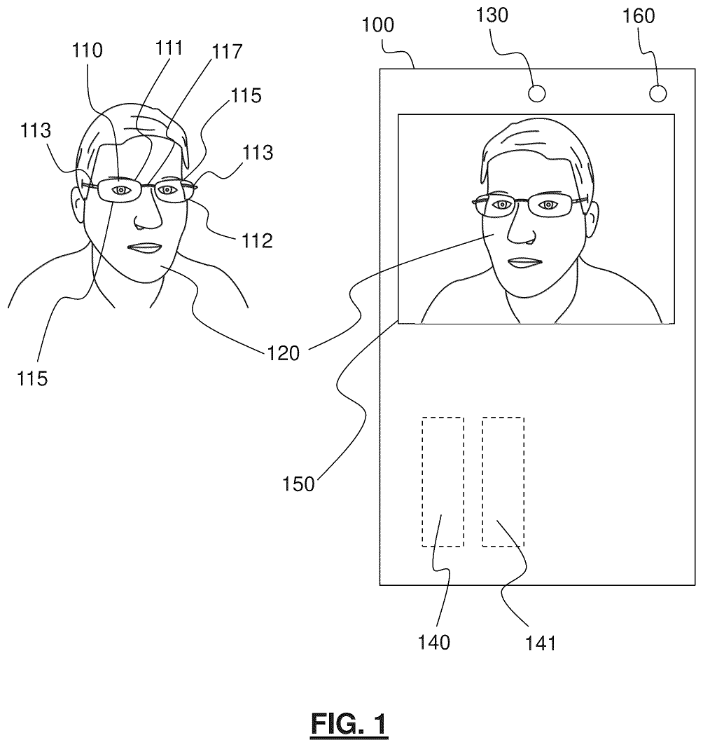

FIG. 1 shows an augmented reality device 100 used by an individual 120 wearing a pair of spectacles 110 on their face 125. The pair of spectacles 110 usually comprises a frame 111 including a front 112 and two arms 113 extending on either side of the face of the individual 120. Furthermore, the front 112 makes possible in particular to carry lenses 114 placed inside the two rims 115 configured in the front 112. Two pads (not shown on FIG. 1) are each secured projecting on the edge of a distinct rim 115 so that they can rest on the nose 121 of the individual 120. A bridge 117 connecting the two rims 115 straddles the nose 121 when the pair of spectacles 110 is worn by the face of the individual 120.

The device 100 comprises a main image-acquisition device, in this case a camera 130, acquiring a plurality of successive images forming a video stream, displayed in real time on a screen 150 of the device 100. A data processor 140 included in the device 100 processes in real time the images acquired by the camera 130 in accordance with the instructions of a method followed according to the disclosure, which are stored in a computer memory 141 of the device 100.

Optionally, the device 100 may also comprise at least one secondary image-acquisition device, in this case at least one secondary camera 160, which may be oriented similarly or differently with respect to the camera 130, making it possible to acquire a second stream of images of the face 125 of the individual 120. In which case it must be emphasized that the position and the relative orientation of the secondary camera 160, or of each secondary camera, with respect to the camera 130 are generally advantageously known.

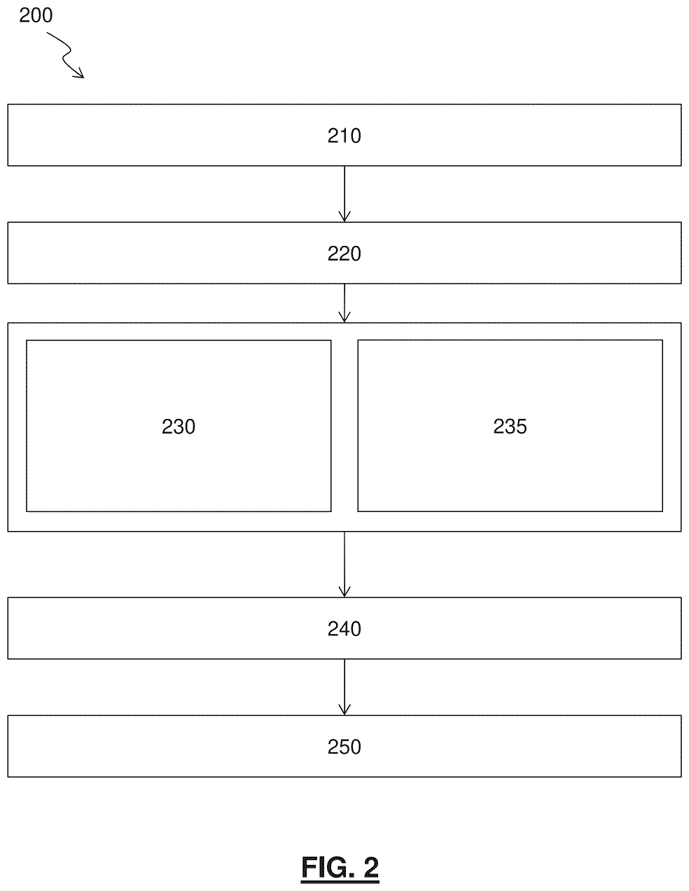

FIG. 2 illustrates, in the form of a block diagram, the method 200 for tracking, in the video stream acquired by the camera 130, the face of the individual 120.

First of all, it must be emphasized that the tracking method 200 is generally implemented in a loop on images, generally successive, of the video stream. For each image, several iterations of each step can be implemented in particular for the convergence of the algorithms used.

The method 200 comprises a first step 210 of detecting the presence of the face of the individual 120 wearing the pair of spectacles 110 in an image of the video stream, referred to as the initial image.

This detection can be implemented in several ways:
either from a learning base of faces wearing a pair of spectacles using a deep learning algorithm, also known by the English term “deep learning”, previously trained on a database comprising images of faces wearing a pair of spectacles;
or by using a three-dimensional model of a face wearing a pair of spectacles that it is sought to make correspond to the image of the face in the initial image by determining a pose, in orientation and in dimension, of the three-dimensional model with respect to the camera 130. The match between the model of the face and the image of the face in the initial image can in particular be made by means of a projection onto the initial image of the model of the face wearing a pair of spectacles. It must be emphasized that this match can be made even if a part of the face or of the pair of spectacles is concealed in the image, as is the case for example when the face is turned with respect to the camera or when elements come to be superimposed on the face, such as a pair of spectacles or hair, or superimposed on the pair of spectacles, such as hair.

Alternatively, the step 210 of detecting in the initial image the face of the individual 120 wearing a pair of spectacles 110 can be implemented by firstly detecting one of the two elements, for example the face, and then secondly the other element, namely here the pair of spectacles. The face is for example detected by means of the detection of characteristic points of the face in the image. Such a method for detecting the face is known to persons skilled in the art. The pair of spectacles can be detected for example by means of a deep learning algorithm, also known by the English term “deep learning”, previously trained on a database of images of a pair of spectacles, preferentially worn by a face.

It must be emphasized that the detection step 210 can be implemented only once for a plurality of images of the video stream.

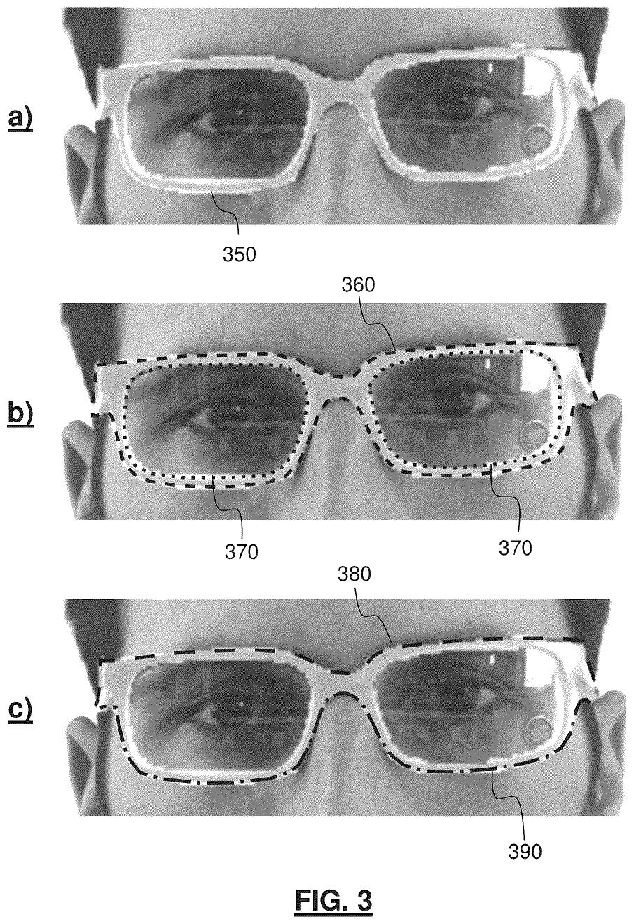

As illustrated on FIG. 3, the learning algorithm makes it possible in particular to calculate a binary mask 350 of the pair of spectacles for each of the images acquired.

The contour points of the mask, denoted p2D, are each associated with at least one category such as:
an exterior contour 360 of the mask;
an interior contour 370 of the mask, generally corresponding to a contour of a lens;
a contour 380 of the top of the mask;
a contour 390 of the bottom of the mask.

Alternatively, the contour points of the mask, p2D, are calculated using a robust distance, i.e. varying little between two successive iterations, between characteristic points of the pair of spectacles detected in the image and contour points of the mask.

After having detected the face of the individual 120 wearing the pair of spectacles 110, the method 200 comprises a second step 220 of alignment of a representation of the face of the individual, hereinafter referred to as an “avatar”, with the image of the face of the individual 120 in the initial image. The avatar here advantageously comprises two parametric models, one corresponding to a model of a face without a pair of spectacles and the other to a model of a pair of spectacles. It must be emphasized that the parametric models are generally placed in a virtual space the origin of the reference frame of which corresponds to the camera 130. The reference frame of the camera will thus be spoken of.

The conjoint use of these two parametric models makes it possible to increase the performance of the regression and to obtain a better estimation of the position of the model of the face of the individual with respect to the camera.

Furthermore, the two parametric models of the avatar are here advantageously linked together by relative orientation and positioning parameters. Initially, the relative orientation and positioning parameters correspond for example to a standard pose of the parametric model of the pair of spectacles with respect to the parametric model of the face, i.e. so that the frame rests on the nose, facing the eyes of the individual, and the arms extend along the temples of the individual resting on the ears of the latter. This standard pose is for example calculated by an average positioning of a pair of spectacles positioned naturally on the face of an individual. It must be emphasized that the pair of spectacles may be advanced on the nose to a greater or lesser extent according to the individuals.

The parametric model of the pair of spectacles is, in the present non-limitative example of the disclosure, a model including a three-dimensional frame the envelope of which includes a non-zero thickness, at least in cross section. Advantageously, the thickness is non-zero in each part of the cross section of the frame.

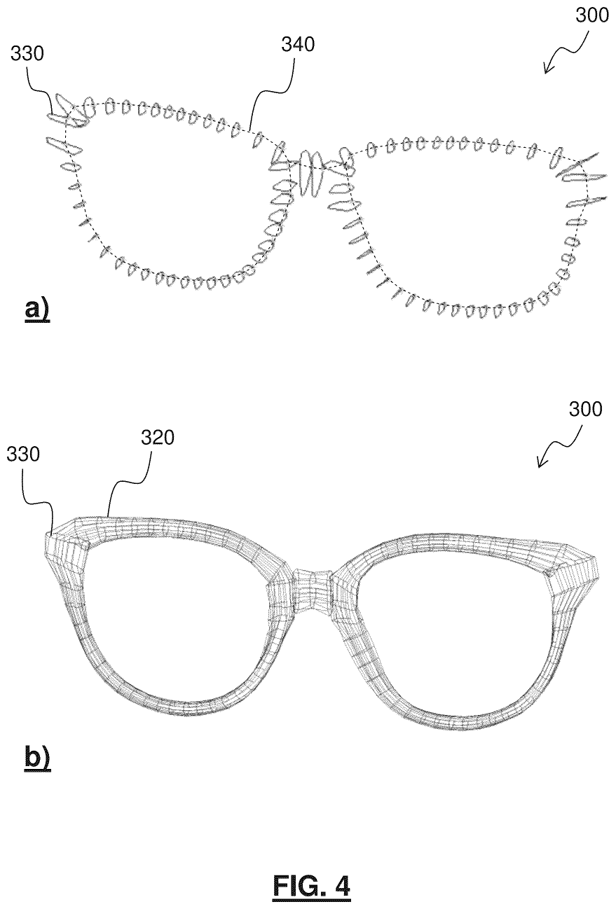

FIG. 4 presents the face 300 of the parametric model of the pair of spectacles in two views. The first view, denoted 4a, corresponds to a view of the skeleton of the face 300, without external envelope. The second view, denoted 4b, corresponds to the same view but with external envelope 320. As illustrated, the parametric model of the pair of spectacles can be represented by a succession of contours 330 each with a cross-section perpendicular to a core 340 of the frame of the pair of spectacles. The contours 330 thus form a skeleton for the external envelope 320. This parametric model is of the 3D type with thickness.

It must be emphasized that the parametric model of the pair of spectacles can advantageously comprise a predetermined number of numbered sections so that the position of the sections around the frame is identical for two distinct models of a pair of spectacles. The section corresponding to the point of the frame, such as a bottom point of a rim, a top point of a rim, a junction point between a rim and the bridge, or a junction point between a rim and a tenon carrying a hinge with an arm, thus has the same number in the two distinct models. It is thus easier to adapt the model of the pair of spectacles to the indications of dimensions of the frame. These indications, normally referred to by the English term “frame marking”, define the width of a lens, the width of the bridge or the length of the arms. This information can then serve in defining constraints between two points, corresponding for example to the center or to the edge of two sections selected according to their position on the frame. The model of the pair of spectacles can thus be modified while complying with the dimension constraints.

An example of a parametric model of the pair of spectacles used by the present method is presented below in more detail in a section entitled “Example of a parametric model of a pair of spectacles”.

In alternative aspects of the disclosure, the parametric model of the pair of spectacles includes a three-dimensional frame of zero thickness. This is then a 3D-type model without thickness.

All the parameters for defining the morphology and size of the pair of spectacles are referred to as configuration parameters.

It must be emphasized that the initial form of the frame of the parametric model can advantageously correspond to the form of the frame of the pair of spectacles that was previously modelled by a method as described for example in the French patent published under the number FR 2955409 or in the international patent application published under the number WO 2013/139814.

The parametric model of the pair of spectacles can also advantageously be deformed, for example at the arms or at the front, which are generally formed from a material able to deform elastically. The deformation parameters are included in the configuration parameters of the model of the pair of spectacles. In the case where the model of the pair of spectacles is known, by means for example of a prior modeling of the pair of spectacles 110, the model of the pair of spectacles can advantageously remain invariant in size and form during the resolution. Only the deformation of the model of the pair of spectacles is then calculated. The number of parameters to be calculated being reduced, the calculation time shorter obtaining a satisfactory result.

To align the two parametric models of the representation of the face with respect to the image of the pair of spectacles and of the face in the initial image, a regression of the points of the parametric models is implemented during the second step 220 so that the parametric models correspond in form, in size, in position and in orientation respectively to the pair of spectacles 110 worn by the individual 120 and to the face of the individual 120.

The parameters of the avatar processed by the regression are thus in the present example non-limitative of the disclosure:
the three-dimensional position of the avatar, i.e. of the set {model of pair of spectacles, model of face};
the three-dimensional orientation of the avatar;
the size of the model of the pair of spectacles;
the size of the model of the face;
the relative three-dimensional position between the model of the pair of spectacles and the model of the face;
the relative three-dimensional orientation between the model of the pair of spectacles and the model of the face;
optionally, configuration parameters of the model of the pair of spectacles;
optionally, configuration parameters of the model of the face such as morphological parameters for defining the form, the size and the position of the various elements constituting a face, such as in particular the nose, the mouth, the eyes, the temples, the cheeks, etc. The configuration parameters can also comprise parameters of opening and closing of the eyelids or of the mouth, and/or parameters related to the deformations of the surface of the face due to expressions;
optionally parameters of the camera, such as a focal length or a metric calibration parameter.

Alternatively, only some of the parameters of the avatar listed above are processed by the regression.

The parameters of the camera can advantageously be calculated when the 3D geometry of the model of the pair of spectacles is known, for example when the pair of spectacles 110 worn by the individual 120 has been recognized. Adjusting the parameters of the camera helps to obtain a better estimation of the parameters of the avatar, and consequently better tracking of the face in the image.

The regression is advantageously implemented here in two stages. Firstly, a minimization of the characteristic points of the model of the face with the characteristic points detected on the initial image is implemented to obtain an estimative position of the avatar in the reference frame of the camera.

Secondly, the parameters of the avatar are refined by implementing a regression of the points of the contour of the model of the pair of spectacles with respect to the pair of spectacles as visible on the initial image of the video stream. The points of the contour of the model of the pair of spectacles considered during the regression generally come from the frame of the pair of spectacles.

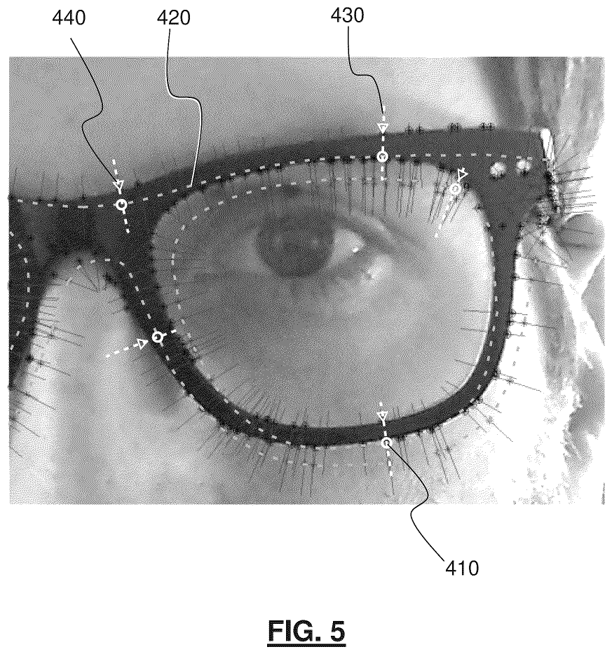

For this purpose, and as illustrated in FIG. 5, the points 410 considered of the contour of the model 420 of the pair of spectacles are those the normal lines 430 of which are perpendicular to the axis between the corresponding point 410 and the camera. A point of the contour of the pair of spectacles on the initial image is associated with each point 410 considered of the contour of the model of the pair of spectacles, seeking the point 440 along the normal line 430 having the highest gradient, for example in a given color spectrum such as in gray level. The contour of the pair of spectacles can also be determined by means of a deep learning method, also known by the English term “deep learning”, previously trained on images of segmented pairs of spectacles, preferentially worn by a face. By minimizing the position between the points of the contours of the model and of the pair of spectacles on the initial image, it is thus possible to refine the parameters of the avatar in the reference frame of the camera.

It must be emphasized that, for reasons of clarity, only five points 410 have been shown on FIG. 5. The number of points used by the regression is generally appreciably higher. The points 410 are represented by a circle on FIG. 4, the points 440 corresponding to a vertex of a triangle sliding along a normal line 430.

The association of a point of the contour of the model of the pair of spectacles with a point of the contour of the pair of spectacles 110 in the image corresponds to a pairing of a 3D point of the model of the pair of spectacles with a 2D point of the image. It must be emphasized that this pairing is preferentially evaluated at each iteration, or even at each image, since the corresponding point in the image may have slipped from one image to the other.

Furthermore, the category or categories of the point of the contour in the image advantageously being known, the pairing of this point with a 3D point of the model of the pair of spectacles can be implemented more effectively by pairing points having the same categories. It must in fact be emphasized that the points of the model of the pair of spectacles can also be classified according to the same categories as the points of the contour of the mask of the pair of spectacles in the image.

In order to improve the regression around the positioning of the model of the pair of spectacles, a contour of a section is advantageously associated with the majority of the points considered of the contour of the model of the pair of spectacles. The section associated with a point generally corresponds to the edge of the frame comprising this point. Each section is defined by a polygon comprising a predetermined number of ridges. Thus, during the regression, the calculation of the normal line is improved by being more precise, which makes it possible to have a better estimation of the pose of the model of the pair of spectacles with respect to the image. This improvement is in particular applicable in the case of the use of a parametric model of the 3D pair of spectacles with thickness.

It must also be emphasized that, during the regression, positioning constraints between the model of the face and the model of the pair of spectacles are advantageously taken into account in order to reduce the calculation time while offering better quality of pose. The constraints indicate for example a collision of points between a part of the model of the face and a part of the model of the pair of spectacles. These constraints represent for example the fact that the rims, via the pads or not, of the pair of spectacles rest on the nose and that the arms rest on the ears. Generally the positioning constraints between the model of the face and the model of the pair of spectacles make it possible to parameterize the positioning of the pair of spectacles on the face with a single parameter, for example the position of the pair of spectacles on the nose of the individual. Between two positions on the nose, the pair of spectacles makes a translation on a 3D curve corresponding to the ridge of the nose, or even a rotation on an axis perpendicular to this symmetry midplane. Locally between two close points, it can be considered that the translation of the pair of spectacles on the 3D curve follows a local symmetry plane of the nose.

In other words, the constraint is represented by a pairing of a point of the model of the face with a point of the model of the pair of spectacles. It must be emphasized that the pairing between two points may be of the partial type, namely relate only to one type of coordinate, which is for example only the X-axis, in order to leave free the translation of one of the two models with respect to the other along the other two axes.

Moreover, each of the two parametric models included in the avatar, i.e. that of the face and that of the pair of spectacles, can also advantageously be constraints on a known dimension such as an interpupillary distance previously measured for the face or a characteristic dimension of the frame previously recognized. A pairing between two points of the same model can thus be implemented to constrain the distance between these two points on the known dimension.

For more mathematical details of the algorithm, reference can be made to the presentation made below in the section entitled “Details of the method implemented”.

It must be emphasized that, when at least one secondary camera is available, several views of the face of the individual wearing the pair of spectacles are available, which makes it possible to improve the regression calculation of the parameters of the avatar. This is because the various views are acquired with a distinct angle, thus making it possible to improve the knowledge of the face of the individual by displaying parts concealed on the image acquired by the main camera.

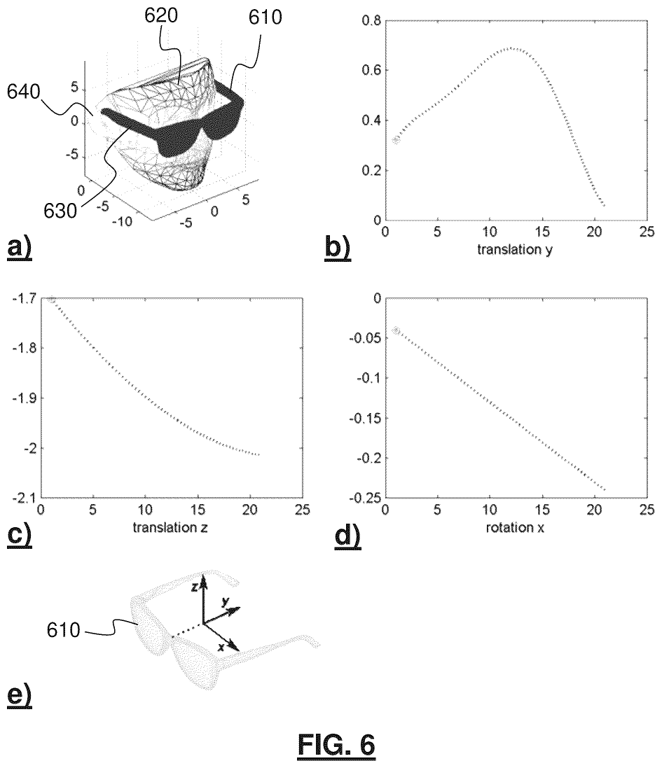

FIG. 6 illustrates the position of the parametric model 610 of the pair of spectacles on the parametric model 620 of the face of the avatar, which is visible in a perspective view in sub-figure a. The reference frame used is illustrated by sub-figure e of FIG. 6. The movement of the parametric model 610 of the pair of spectacles is here parameterized according to a movement of the arms 630 on the ears 640, corresponding to the translation along the Z-axis (sub-figure c of FIG. 6). The translation along the corresponding Y-axis is visible on sub-figure b of FIG. 6. The rotation about the X-axis is illustrated on sub-figure d of FIG. 6.

Constraints of non-collision between certain parts of the model of the face and certain parts of the model of the pair of spectacles can also be added in order to avoid faulty positioning of the model of the pair of spectacles on the model of the face, for example an arm in an eye of the individual, etc.

One difficulty surmounted by the present disclosure is the management of the concealed parts of the pair of spectacles in the initial image, which can cause errors in the regression of the parametric model of the pair of spectacles, in particular with regard to the position and orientation of the parametric model with respect to the pair of spectacles 110 actually worn by the individual 120. These concealed parts generally correspond to parts of the frame that are masked either by the face of the individual, for example when the face is turned with respect to the camera in order to see a profile of the face, or directly by the pair of spectacles, for example by tinted lenses. It must also be emphasized that the part of the arms placed on each ear is generally obscured, whatever the orientation of the face of the individual 120, by an ear and/or by hair of the individual 120.

These concealed parts can for example be estimated during detection by considering a segmentation model of the frame and/or points of the contour of these concealed parts. The concealed parts of the pair of spectacles can also be estimated by calculating a pose of a parametric model of a pair of spectacles with respect to the estimated position of the face of the individual 120. The parametric model used here can be the same as that used for the avatar.

The alignment of the parametric model of the pair of spectacles also makes it possible to recognize the model of the pair of spectacles 110 actually worn by the individual 120. This is because the regression of the points makes it possible to obtain an approximate 3D contour of at least a part of the pair of spectacles 110. This approximate contour is next compared with the contours of pair of spectacles previously modeled, recorded in a database. The image included in the contour can also be compared with the appearance of pairs of spectacles recorded in the database for better recognition of the model of the pair of spectacles 110 worn by the individual 120. It must in fact be emphasized that the models of pairs of spectacles stored in the database have generally been modeled in texture and in material.

The parametric model of the pair of spectacles can be deformed and/or articulated in order best to correspond to the pair of spectacles 110 worn by the individual 120. Generally, the arms of the model of the pair of spectacles initially form between them an angle of the order of 5°. This angle can be adjusted by modeling the deformation of the pair of spectacles according to the form of the frame and the rigidity of the material used for the arms, or even also the material used for the front of the frame of the pair of spectacles, which may be distinct from that of the arms. A parametric approach can be used for modeling the deformation of the parametric model of the pair of spectacles.

A real-time tracking of the face and/or of the pair of spectacles in the video stream, on images successive to said initial image, is implemented during a third step 230 of the method 200 illustrated in FIG. 2.

The real-time tracking can for example be based on the tracking of characteristic points in successive images of the video stream, example using an optical flow method.

This tracking can in particular be implemented in real time since the updating of the parameters for an image of the video stream is generally implemented with respect to the alignment parameters calculated at the previous image.

In order to improve the robustness of the tracking, the use of key images, usually referred to by the English term “keyframe”, where the pose of the avatar with respect to the face of the individual is considered to be satisfactory, can be used for providing constraints on the images presenting views of the face oriented in a similar manner to the face in a key image. In other words, a key image of a selection of images of the video stream, which can also be referred to as a reference image, generally corresponds to one of the images of the selection where the score associated with the pose of the avatar with respect to the image of the individual is the highest. Such a tracking is for example described in detail in the international patent application published under the number WO 2016/135078.

It must be emphasized that the selection of a key image can be made dynamically and that the selection of images can correspond to a continuous sequence of the video stream.

Furthermore, the tracking can advantageously use a plurality of key images, each corresponding to a distinct orientation of the face of the individual.

It must also be emphasized that the conjoint tracking of the face and of the pair of spectacles makes it possible to obtain better, more robust, results since they are based on a larger number of characteristic points. Furthermore, the relative positioning constraints of the parametric models of the face and of the pair of spectacles are generally used during the trackingtracking, which makes it possible to obtain a more precise trackingtracking of the head of the individual in real time, and consequently a better pose of the avatar.

Moreover, the tracking of the pair of spectacles, which is a manufactured object, is generally more precise than the tracking of a face alone, since the pair of spectacles includes landmarks that are clearly identifiable in an image, such as a ridge of an arm, a ridge of the face or a rim of the front of the frame.

It must be emphasized that a tracking of the pair of spectacles, without the use of a parametric model of the pair of spectacles, would be less robust and would require a large number of calculations for each image. Such a tracking is thus more difficult to implement in real time having regard to the computing powers currently available. However, because of the regular increase in the power of processors, tracking without use of a parametric model of the pair of spectacles could be envisaged when the powers of the processors are sufficient for such an application.

It must also be emphasized that it is possible to implement a tracking of the individual on the basis solely of the parametric model of the pair of spectacles. Optimization of the pose of the model of the pair of spectacles with respect to the camera, i.e. the alignment of the model of the pair of spectacles with respect to the image, is implemented for each image.

An updating of the alignment parameters of the parametric models of the face and of the pair of spectacles with the image is next implemented for each new image of the video stream acquired by the camera 130, concomitantly with the tracking step 230, during a step 235.

Alternatively, the updating of the alignment parameters of the parametric models of the face and of the pair of spectacles is implemented at each key image.

This updating of the alignment parameters can also comprise the parameter of pose of the parametric model of the pair of spectacles on the parametric model of the face, in order to improve the estimation of the positioning of the face of the individual with respect to the camera. This updating can in particular be implemented when the face of the individual is oriented differently with respect to the camera, thus offering another angle of view of their face.

A refinement of the parametric models can be implemented during a fourth step 240 of the method 200 by analyzing the reference key images used during the tracking. This refinement makes it possible for example to complete the parametric model of the pair of spectacles with details of the pair of spectacles 110 that had not been captured previously. These details are for example a relief, an aperture or a serigraphy specific to the pair of spectacles.

The analysis of the key images is done by a cluster adjustment method, also known by the English term “bundle adjustment”, which makes it possible to refine the 3D coordinates of a geometric model describing an object of the scene, such as the pair of spectacles or the face. The “bundle adjustment” method is based on a minimization of the re-projection errors between the observed points and the points of the model.

Thus it is possible to obtain parametric models more conforming to the face of the individual wearing the pair of spectacles.

The analysis by the “bundle adjustment” method here uses characteristic points of the face and points of the spectacles that are identifiable with more precision in the key image. These points can be points of the contour of the face or of the spectacles.

It must be emphasized that the “bundle adjustment” method in general terms processes a scene defined by a series of 3D points that can move between two images. The “bundle adjustment” method makes it possible to simultaneously solve the three-dimensional position of each 3D point of the scene in a given reference frame (for example that of the scene), the parameters of relative movements of the scene with respect to the camera and the optical parameters of the camera or cameras that acquired the images.

Sliding points calculated by means of an optical flow method, for example related to the points of the contour of the face or spectacles, can also be used by the “bundle adjustment” method. However, the optical flow being calculated between two distinct images, generally consecutive in the video stream, or between two key images, the matrix obtained during the “bundle adjustment” method for the points coming from the optical flow is generally hollow. To compensate for this lack of information, points of the contour of the spectacles can advantageously be used by the “bundle adjustment” method.

It must be emphasized that new information making it possible to improve the parametric model of the face or the parametric model of the pair of spectacles can be obtained for a new key image. Furthermore, a new detection of the face wearing the pair of spectacles, such as the one described in step 210, can be implemented in this new key image, in order to supplement or replace the points used by the “bundle adjustment” method. A resolution constraint with higher weight can be associated with the new points detected in order to ensure that the refinement of the parametric models is closer to the current image of the video stream.

Sliding points of the contour of the spectacles can be paired with the 3D model of the pair of spectacles on a level line of the contour of the spectacles, corresponding to all the points of the model of the pair of spectacles the normal of which is at 90 degrees.

In an example of an aspect of the disclosure, the key images correspond to images when the face of the individual 120 wearing the pair of spectacles 110 is face on, and/or to images where the face of the individual 120 is turned to the left or to the right with respect to the natural position of the head by an angle of the order of 15 degrees with respect to the sagittal plane. For these key images, new parts of the face 125 and of the pair of spectacles 110 are visible. The parameters of the models of the face and of the pair of spectacles can thus be determined with more precision. The number of key images can be fixed arbitrarily at a number lying between 3 and 5 images in order to obtain satisfactory results in the learning of the face 125 and of the pair of spectacles 110 for establishing the corresponding models.

The size of the pair of spectacles 110 worn by the individual 120 can also be introduced during the method 200 in a step 250, in particular to obtain a metric of the scene, and to define a scale in particular for determining an optical measurement of the face of the individual, such as for example an interpupillary distance or a size of an iris, which can be defined as a mean size.

The size of the pair of spectacles 110 can be defined statistically with respect to a list of pairs of spectacles previously defined, or correspond to the actual size of the pair of spectacles 110.

An interface can be provided for indicating to the method 200 which is the “frame marking” indicated in the pair of spectacles 110. Alternatively, an automatic reading on an image can be done by the method 200 for recognizing the characters of the “frame marking” and automatically obtaining the associated values.

It must be emphasized that, when the “frame marking” is known, the parametric model of the pair of spectacles 110 can advantageously be known, in particular if the pair of spectacles 110 was previously modelled.

When no size information on the pair of spectacles is available, for example when the “frame marking” is unknown, the parametric model of the pair of spectacles used initially is a standard parametric model comprising statistically mean values of the pairs of spectacles normally used by individuals. This statistical framework makes it possible to obtain a satisfactory result, close to the model of the pair of spectacles 110 actually worn by the individual 120, each new image improving the parameters of the model of the pair of spectacles.

A depth camera can also be used during the method 200 in order to refine the form and position of the face.

It must be emphasized that the depth camera is a type of depth sensor, usually known by the English term “depth sensor”. Furthermore, the depth sensor, generally operating using the emission of an infrared light, is not sufficiently precise to acquire the contours of the pair of spectacles 110 worn by the individual 120, in particular because of the refraction, transmission and/or reflection problems introduced by the lenses and/or the material of a front of the pair of spectacles. In some cases, light conditions, such as the presence of an intense light source in the field of the camera, prevent the correct operation of the infrared depth camera by introducing high noise preventing any reliable measurements. However, the depth measurements can be used on visible parts of the face, in order to guarantee depth measurements on the visible surface of the face, the metric and better estimation of the size and form of the model of the face or even also of the model of the pair of spectacles.

Provided that the face of the individual 120, or at least only of the pair of spectacles 110, is tracked by the previously described method 200, a deletion of the pair of spectacles 110 worn by the individual 120 in the video stream can be implemented by referring in particular to the technique described in the international patent application published under the number WO 2018/002533. A virtual try-on of a new pair of spectacles can furthermore be implemented.

It must be emphasized that, the tracking method 200 being more effective, the deletion of the pair of spectacles in the image by obscuring the pair of spectacles worn is done more realistically since the position of the pair of spectacles is determined more precisely with respect to the camera by the present tracking method.

It is also possible to modify all or part of the pair of spectacles worn by the individual by virtue of the tracking method described here, for example by changing of color or shade of the lenses, adding an element such as serigraphy, etc.

The tracking method 200 can thus be included in an augmented reality method.

It must be emphasized that the tracking method 200 can also be used in a method for measuring an optical parameter, such as the one described in the international patent application published under the number WO 2019/020521. By using the tracking method 200, the measurement of an optical parameter can be more precise since the parametric models of the pair of spectacles and of the face are resolved conjointly in one and the same reference frame, which is not the case in the prior art where each model is optimized independently without taking account of the relative positioning constraints of the model of the pair of spectacles and of the model of the face.

### Details of the Method Used

The algorithm presented in the present section corresponds to a generic implementation of a part of a tracking method that is the object of the previously detailed example. This part corresponds in particular to the resolving of the parameters, in particular of pose and configuration/morphology, of the model of the face and of the model of the pair of spectacles with respect to points detected in at least one stream of images (step 220 above) and to updating thereof (step 235 above). It must be emphasized that these two steps are generally based on the same equation solved under constraint. The morphological modes of the model of the face and of the model of the pair of spectacles can also be resolved during this part.

The advantage in resolving at the same time the model of the face and the model of the pair of spectacles is to provide new collision or proximity constraints between the model of the face and the model of the pair of spectacles. This is because it is thus ensured firstly that the two meshes, each corresponding to a distinct model, do not interpenetrate each other but also that there are at least points that are in collision, or in proximity, between the two meshes, in particular at the ears and nose of the individual. It must be emphasized that one of the major problems in resolving the pose of a model of the face corresponds to the positionings of the points at the temples, the location of which is rarely determined precisely by the point detector normally used. Use of the arms of the spectacles, which are often much more visible in the image and physically against the temples, is consequently advantageous.

It must be emphasized that it is difficult to establish a collision algorithm within a minimization since the two models used are parametric models, and consequently deformable. Since the two models deform at each iteration, the contact points can then be distinct from one iteration to the other.

In the present non-limitative example of the disclosure, n calibrated cameras are considered, each acquiring p views, namely p images. It must be emphasized that the intrinsic parameters of each camera and the relative positions thereof are known. The position and orientation of the face is nevertheless to be determined for each of the views. The 3D parametric model of the face used, denoted Mf, is a mesh composed of 3D points p3D linearly deformable by means of v parameters denoted α_{k,k=1 . . . v}. Thus each 3D point of this mesh is written in the form of a linear combination:

$${p3D\_f}_{j} ( {α}_{1} , … , {α}_{v} ) = {m3D\_f}_{j} + {\sum }_{k = 1}^{v} {α}_{k} {mode\_f}_{j}^{k} \qquad [ Math 1 ]$$

where m3Dⱼ designates the j^{th} mean point of the model and modeⱼ^{k} the j^{th} vector of the k^{th} mode of the model. The index _f is added to m3Dⱼ, p3D and modeⱼ^{k} to indicate that the model used is that of the face. A similar equation for the model of the pair of spectacles denoted M_{g} can be written:

$${p3D\_g}_{j} ( {β}_{1} , {β}_{2} , … , {β}_{μ} ) = {m3D\_g}_{j} + {\sum }_{k = 1}^{μ} {β}_{k} {mode\_g}_{j}^{k} \qquad [ Math 2 ]$$

where β_{k,k=1 . . . μ} corresponds to μ parameters of the parametric model of the pair of spectacles M_{g}.

The 3D face is replaced initially in a three-dimensional reference frame, referred to as a world reference frame, for each of the p acquisitions. The world reference frame can for example correspond to the reference frame of the camera or to a reference frame of one of the two models. The positions and orientations of the model of the face are initially unknown and consequently sought during the minimization, which corresponds to a phase of regression of the points of the model of the face with characteristic points detected in the image.

Before implementing this regression, the model M_{g} of the pair of spectacles is positioned on the model Mf of the face. For this purpose, the points p3D_g of the model of the pair of spectacles can be written in the reference frame of the face while taking account of a 3D rotation matrix R_g and of a translation vector T_g.

$$p3D\_g {\_p}_{j} = [ \begin{matrix}R\_g & T\_g \\ 000 & 1\end{matrix} ] {p3D\_g}_{j} ( {β}_{1} , {β}_{2} , … , {β}_{μ} ) \qquad [ Math 3 ]$$

The regression next results in a pose in orientation and in translation of the model of the face in the reference frame in the reference frame of the view l of one of the cameras, corresponding here to the world reference frame.

$${p3D\_f}_{j}^{l} = [ \begin{matrix}{R\_f}_{l} & {T\_f}_{l} \\ 000 & 1\end{matrix} ] {p3D\_f}_{j} ( {α}_{1} , … , {α}_{v} ) \qquad [ Math 4 ]$$

where R represents a 3D rotation matrix, T a translation vector and l a view of a camera.

A function of projection of a model p3D in the image i used during the method is denoted:

$${Proj}^{i} ( p 3 D ) ∼ {K}^{i} [ {R}^{i} {T}^{i} ] p 3 D \qquad [ Math 5 ]$$

where Kⁱ corresponds to the calibration matrix of the image i. Rⁱ and Tⁱ correspond respectively to a rotation matrix and to a translation vector between the world reference frame and the reference frame of the camera that acquired the image i. The symbol ˜ for its part designates an equality to within a scale factor. This equality can in particular be represented by the fact that the last component of the projection is equal to 1.

When the pose of the models of the representation of the face is resolved, there are five types of constraint:
the 2D face constraints;
the 2D spectacle constraints;
the 3D face—spectacles constraints;
the 3D face constraints, corresponding for example to an interpupillary distance PD, to a distance between the temples, to a mean iris size or to a mixture of distributions of several size constraints. A mixture of distributions may correspond to a mixture of two Gaussian distributions around the size of an iris and the interpupillary distance. Combining these constraints may have recourse to a formulation of the g-h filter type;
the 3D constraints of the spectacles, corresponding for example to a known dimension resulting from the marking on the frame, normally referred to by the English term “frame marking”.

The 2D constraints of the face are based on a pairing of the points of the 3D model with 2D points in the image of the face for at least one viewer and for at least one camera. Preferentially, this pairing is done for each view and for each camera. It must be emphasized that the pairings can be fixed for the points of the face not included on the contour of the face in the image or sliding along level lines for the points of the contour of the face. This degree of freedom in the pairing of a point of the contour of the face with a point of the image makes it possible in particular to improve the stability of the pose of the 3D model of the face with respect to the image, thus offering better continuity of pose of the 3D model of the face between two successive images.

The pairing of a point of the 3D model of the face with a 2D point of the image can be represented mathematically by the following equation:

$$p 3 {D}_{{f}_{{φ}_{j , i , l}}} ↔ p 2 {D}_{{f}_{{σ}_{j , i , l}}} \qquad [ Math 6 ]$$

where φ_{j,i,l} and σ_{j,i,l} represent respectively an index of a 3D point of the parametric model Mf of the face and an index of a 2D point of the face in the images for a view i and a camera l.

The 2D constraints of the spectacles are based on a pairing of the 3D points of the model of the pair of spectacles with 2D points of the spectacles in an image using in particular the contours of the masks in the images.

$${p3D\_g}_{{θ}_{j , i , l}} ↔ {p2D\_g}_{{ω}_{j , i , l}} \qquad [ Math 7 ]$$

where θ_{j,i,l} and ω_{j,i,l} represent respectively an index of a 3D point of the parametric model Mg of the pair of spectacles and an index of a 2D point of the pair of spectacles in the images for a view i and a camera I.

The 3D face—spectacles constraints are based on a pairing of the 3D points of the model of the face and 3D points of the model of the pair of spectacles, the distance of which is defined by a proximity, or even collision (zero distance), constraint. An influence function can be applied to calculate the collision distance with for example a greater weight for the negative distances with respect to the normal to the surface of the model of the face oriented towards the outside of the model of the face. It must be emphasized that, for some points, the constraint may be solely on some of the coordinates, such as for example on an axis for the relationship between the temples of the face and the arms of the pair of spectacles.

The pairing of the 3D points of the model of the face and 3D points of the model of the pair of spectacles can be represented mathematically by the following equation:

$${p3D\_f}_{{ρ}_{j}} ↔ {p3D\_g}_{{τ}_{j}} \qquad [ Math 8 ]$$

where ρⱼ and τⱼ represent respectively an index of a 3D point of the parametric model Mf of the face and an index of a 3D point of the parametric model Mg of the pair of spectacles.

The 3D constraints on the face are based on a known distance of the face, previously measured, such as for example the interpupillary distance (distance between the center of each pupil, also corresponding to the distance between the center of rotation of each eye). A metric distance can thus be paired with a pair of points.

$$( {p3D\_f}_{{t}_{j}} , {p3D\_f}_{{u}_{j}} ) ↔ {dist}_{{t}_{j} {u}_{j}} \qquad [ Math 9 ]$$

where tⱼ and uⱼ each represent an index of a distinct 3D point of the parametric model Mf of the face.

The 3D constraints on the pair of spectacles are based on a known distance of the model of the pair of spectacles worn by the individual, such as the size of a lens (for example in accordance with the BOXING standard or the DATUM standard), the size of the bridge or the size of the arms. This distance can in particular be represented by the marking of the frame, generally located inside an arm, normally referred to as “frame marking”. A metric distance can then be paired with a pair of points of the model of the pair of spectacles.

$$( {p3D\_g}_{{v}_{j}} , {p3D\_g}_{{w}_{j}} ) ↔ {dist}_{{v}_{j} {w}_{j}} \qquad [ Math 10 ]$$

where vⱼ and wⱼ each represent an index of a distinct 3D point of the parametric model Mg of the pair of spectacles.

The input data of the algorithm are thus:
p images coming from n cameras of a person wearing a pair of spectacles;
characteristic 2D points of the face, detected in an image;
2D or 3D pairings for some of the points, optionally evaluated at each iteration in the case of the so-called sliding points (e.g.: along the level lines);
the mask of the pair of spectacles in at least one image;
the calibration matrix and the pose of each camera.

The algorithm will make it possible to calculate the following output data:
the p poses of the avatar: R_{ft}, T_{ft};
the v modes of the parametric model of the face: α₁, α₂, . . . , αₙ;
the pose of the model of the pair of spectacles with respect to the model of the face: R_{g}, T_{g};
the μ modes of the parametric model of the pair of spectacles: β₁, β₂, . . . β_{μ}.

For this purpose, the algorithm proceeds in accordance with the following steps:
implementing the pairings of the points φ_{j,i,l}↔σ_{j,i,l} for the 2D constraints of the face;
implementing the pairings of the points θ_{j,i,l}↔ω_{j,i,l} for the 2D constraints of the pair of spectacles;
implementing the pairings of the points ρⱼ↔τⱼ for the 3D constraints between the model of the face and the model of the pair of spectacles;
implementing the pairings of the points tⱼ↔uⱼ and associating them with a metric distance distₜ_{ⱼ}ᵤ_{ⱼ} to establish the 3D constraints on the model of the face;
implementing the pairings of the points vⱼ↔wⱼ and associating them with a metric distance distᵥ_{ⱼ}_{w}_{ⱼ} to establish the 3D constraints on the model of the pair of spectacles;
solving the following mathematical equation.

$$[ Math 11 ]$$

$$\text{?} \frac{{γ}_{1}}{\# ( visi == 1 )} ( {\sum }_{i = 1}^{n} {\sum }_{l = 1}^{p} {\sum }_{j = 1}^{{m}_{1}} visi ( p2D\_ \text{?} ) {‖ {Proj}^{i} ( [ \begin{matrix}{R}_{{f}_{l}} & {T}_{{f}_{l}} \\ 000 & 1\end{matrix} ] p3D\_ \text{?} ( {α}_{1} , … , {α}_{v} ) - p2D\_ \text{?} ‖}_{2} ) + \frac{{γ}_{2}}{\# ( visi == 1 )} ( {\sum }_{i = 1}^{n} {\sum }_{l = 1}^{p} {\sum }_{j = 1}^{{m}_{2}} visi ( p2D\_ \text{?} ) {‖ {Proj}^{i} ( [ \begin{matrix}{R}_{{f}_{l}} & {T}_{{f}_{l}} \\ 000 & 1\end{matrix} ] [ \begin{matrix}R\_g & T\_g \\ 000 & 1\end{matrix} ] p3D\_ \text{?} ( {β}_{1} , {β}_{2} , … , {β}_{μ} ) ) - p2D\_ \text{?} ‖}_{2} ) + {γ}_{3} ( \text{?} {‖ {p3D\_f}_{{ρ}_{j}} ( {α}_{1} , … , {α}_{v} ) [ \begin{matrix}R\_g & T\_g \\ 000 & 1\end{matrix} ] p3D\_ \text{?} ( {β}_{1} , {β}_{2} , … , {β}_{μ} ) ‖}_{2} ) + {γ}_{4} ( {\sum }_{j = 1}^{{m}_{4}} {‖ {‖ {p3D\_f}_{{t}_{j}} ( {α}_{1} , … , {α}_{v} ) - p3D\_ \text{?} ( {α}_{1} , … , {α}_{v} ) ‖}_{2} - dis \text{?} ‖}_{2} ) + {γ}_{5} ( \text{?} {‖ {‖ {p3D\_g}_{{v}_{j}} ( {β}_{1} , {β}_{2} , … , {β}_{μ} ) - p3D\_ \text{?} ( {β}_{1} , {β}_{2} , … , {β}_{μ} ) ‖}_{2} - dis \text{?} ‖}_{2} )$$

$$\text{?} \text{indicates text missing or illegible when filed}$$

where γ₁, γ₂, γ₃, γ₄, γ₅ are weights between each constraint block, visi is a function indicating whether a point p2D is visible in the image, i.e. not obscured by the model of the face Mf or by the model of the pair of spectacles Mg, #(visi==1) corresponds to the number of points visible.

In variants of this particular aspect of the disclosure, the focal length of the camera forms part of the parameters to be optimized. This is because, in the case where the acquisition of the images is done by an unknown camera, some images acquired are previously reframed or resized. In which case it is preferable to leave the focal length of the camera as a degree of freedom during the minimization.

In variants of this particular aspect of the disclosure, the variance and covariance matrices that represent the axes and uncertainty/confidence values of the parameters for the equations of collision constraints between the model of the face and the model of the pair of spectacles are taken into account in the solving.

In variants of this particular aspect of the disclosure, some parameters of the pose of the model of the pair of spectacles with respect to the model of the face are fixed. This may represent a hypothesis of alignment between the model of the pair of spectacles and the model of the face. In this case, only the rotation on the X axis, i.e. on an axis perpendicular to the sagittal plane, and the translation along y and z, i.e. in the sagittal plane, are calculated. The cost function, represented by [Math 11], can be simplified, which makes it possible to obtain an easier convergence towards the result. In this way, it is also possible to obtain very satisfactory results for highly asymmetric faces where the pair of spectacles may be positioned differently compared with a symmetric face, for example slightly inclined on one side of the face.

### Example of a Parametric Model of a Pair of Spectacles

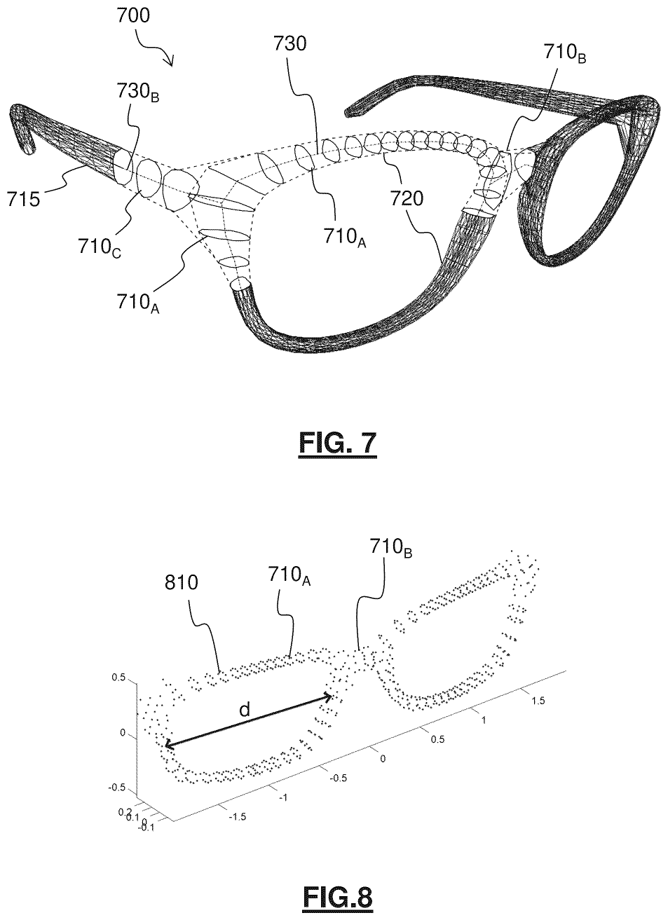

Each pair of spectacles includes common elements such as the lenses, the bridge and the arms. A parametric model (3DMM) 700 of a pair of spectacles, as shown in FIG. 7, can thus be defined as a set of sections 710 connected together by triangular faces 715 previously defined.

The triangular faces 715 form a convex envelope 720, part of which is not shown on FIG. 7.

Each of the sections 710, defined by the same number of points, is advantageously located at the same place on all the models of a pair of spectacles.

Furthermore, each section 710 intersects the pair on a plane perpendicular to the skeleton 730.

Three types of section can thus be defined:
the sections 710_{A} around the lenses, parameterized for example by an angle with respect to a reference plane perpendicular to the skeleton of a rim, in order to have one section every n degrees;
the sections 710 of the bridge, parallel to the reference plane;
the sections 710_{C} of the arms, along the skeleton 730_{B} of the arms.

It must be emphasized that, in the case of a pair without a rim around a lens, usually referred to by the English term “rimless”, or in the case of a pair referred to as “semi-rimless”, i.e. a rim surrounds only a part of a lens, all or some of the sections 710_{A} around the lenses have just a single point corresponding to the combination of all the points of one and the same section 710_{A}.

Moreover, the principal-component analysis (PCA) used in the alignment of the model 700 of the pair of spectacles with the representation of the pair of spectacles in the image requires a number of common points. For this purpose, points that are located on the convex envelope 720 of the model of the pair of spectacles are selected in order to ensure that all the pixels belonging to the aligned pair of spectacles are found in the image.

To make it possible to find apertures in the pair of spectacles, such as for example in the case of a pair of spectacles having a double bridge, a template of the model of a pair of spectacles, for example with a double bridge, can be selected in advance to adapt to the pair of spectacles as closely as possible.

Since a point of the parametric model, referenced with a given index, is continuously located at the same relative point on the model of the pair of spectacles, a definition of the known distance between two points can be facilitated. This known distance can be obtained by the “frame marking” inscribed on a pair of spectacles, which defines the width of the lenses, the width of the bridge all the length of the arms.

This information can then be imposed in the resolution of the spectacles model 700 by selecting the corresponding points, as illustrated by FIG. 8. In FIG. 8, only the points 810 characterizing the contours of the sections 710 of the front of the pair of spectacles are shown, and d corresponds to the width of a lens as defined by means in particular of the “frame marking”.

In a variant of the face and spectacles alignment, a large number of faces and a large number of spectacles are generated from two respective parametric models of the face and of the pair of spectacles. The automatic positioning algorithm is next used for positioning each model of a pair of spectacles on each face model. Advantageously a noise generation and different positioning statistics—spectacles at the end of the nose, recessing of the pads, loose positioning on the temples, etc—are used for automatically positioning the pairs of spectacles on the faces. A new parametric model for the pair of spectacles and for the face is next calculated from all the points of the models of the face and of the pair of spectacles. This new parametric model guarantees collision and perfect positioning of the pair of spectacles on the face, which simplifies the resolution. This is because a single transformation is sought, which corresponds to the calculation of six parameters instead of twelve, and the collision equations are withdrawn. However, a larger number of modes are generally estimated in this case since it is they that encode these constraints.

What is claimed is:

1. Method for tracking a face of an individual in a video stream acquired by an image-acquisition device, the face wearing a pair of spectacles, the video stream comprising a plurality of images acquired successively, characterized in that the tracking method comprises a step of evaluating parameters of a representation of the face comprising a model of the pair of spectacles and a model of the face so that said representation of the face is superimposed on the image of the face in the video stream, wherein all or some of the parameters of the representation are evaluated by taking account of at least one proximity constraint between at least one point of the model of the face and at least one point of the model of the pair of spectacles.

2. Tracking method according to claim 1, wherein the parameters of the representation comprise values external to the representation of the face and values internal to the representation of the face, the external values comprising a three-dimensional position and a three-dimensional orientation of the representation of the face with respect to the image-acquisition device, the internal values comprising a three-dimensional position and a three-dimensional orientation of the model of the pair of spectacles with respect to the model of the face, said parameters being evaluated with respect to a plurality of characteristic points of said representation of the face, previously detected in an image of the video stream, referred to as first image, or in a set of images acquired simultaneously by a plurality of image-acquisition devices, the set of images comprising said first image.

3. Tracking method according to claim 1, wherein all or some of the parameters of the representation are updated with respect to the position of all or some of the characteristic points, tracked or detected, in a second image of the video stream or in a second series of images acquired simultaneously by the plurality of image-acquisition devices, the second set of images comprising said second image.

4. Tracking method according to claim 1, wherein, in evaluating all or some of the parameters of the representation, account is also taken of at least one proximity constraint between a three-dimensional point of one of the models included in the representation of the face and at least one point, or a level line, included in at least one image of the video stream.

5. Tracking method according to claim 1, wherein, in evaluating all or some of the parameters of the representation, account is also taken of at least one dimension constraint of one of the models included in the representation of the face.

6. Tracking method according to claim 1, wherein the method comprises a step of pairing two distinct points belonging either to one of the two models included in the representation of the face, or each to a distinct model from the models included in the representation of the face.

7. Tracking method according to claim 1, wherein the method comprises a prior step of pairing a point of one of the two models included in the representation of the face with at least one point of an image acquired by an image-acquisition device.

8. Tracking method according to claim 1, wherein, during the evaluation of the parameters of the representation, an alignment of the model of the pair of spectacles with an image of the pair of spectacles in the video stream is implemented consecutively with an alignment of the model of the face with an image of the face in the video stream.

9. Tracking method according to claim 8, wherein the alignment of the model of the face is implemented by minimizing the distance between characteristic points of the face detected in the image of the face and characteristic points of the model of the face projected in said image.

10. Tracking method according to claim 8, wherein the alignment of the model of the pair of spectacles is implemented by minimizing the distance between at least a part of the contour of the pair of spectacles in the image and a similar contour part of the model of the pair of spectacles projected in said image.

11. Tracking method according to claim 1, wherein the parameters of the representation comprise all or part of the following list:
a three-dimensional position of the representation of the face;
a three-dimensional orientation of the representation of the face;
a size of the model of the pair of spectacles;
a size of the model of the face;
a relative three-dimensional position between the model of the pair of spectacles and the model of the face;
a relative three-dimensional orientation between the model of the pair of spectacles and the model of the face;
one or more parameter(s) of the configuration of the model of the pair of spectacles;
one or more parameter(s) of the configuration of the model of the face;
one or more parameter(s) of the camera.

12. Tracking method according to claim 11, comprising steps of:
detection of a plurality of points of the face in a first image of the video stream;
initialization of the set of parameters of the model of the face with respect to the image of the face in said first initial image;
detection of a plurality of points of a pair of spectacles worn by the face of the individual in a second image of the video stream, referred to as second initial image, the second initial image being either subsequent to or prior to the first initial image in the video stream, or identical to the first image in the video stream;
initialization of the set of parameters of the model of the pair of spectacles with respect to the image of the pair of spectacles in said second initial image.

13. Tracking method according to claim 12, wherein the initialization of the parameters of the model of the face is implemented by means of a deep learning method of analyzing all or some of the detected points of the face.

14. Tracking method according to claim 13, wherein the deep learning method also determines the initial position of the model of the face in the three-dimensional reference frame.

15. Tracking method according to claim 1, also comprising a step of determining a scale of the image of the pair of spectacles worn by the face of the individual by means of a dimension in the image of an element of known size of the pair of spectacles.

16. Tracking method according to claim 15, wherein the scale is determined by means of a prior recognition of the pair of spectacles worn by the face of the individual.

17. Tracking method according to claim 1, wherein the images acquired by a second image-acquisition device are used to evaluate the parameters of the representation.

18. Tracking method according to claim 1, wherein the model of the pair of spectacles of the representation corresponds to a prior modelling of said pair of spectacles, and varies solely in deformation.

19. Augmented reality method comprising steps of:
acquiring at least one stream of images of an individual wearing a pair of spectacles on their face by means of at least one image-acquisition device;
tracking of the face of the individual by a tracking method according to claim 1, a position and an orientation of a representation of the face;
modifying of all or some of the images of said or of one of said image streams, referred to as main video stream, acquired by the image-acquisition device or by one of the image-acquisition devices, referred to as main image-acquisition device, by means of the representation of the face superimposed in real time on the face of the individual on the main video stream;
displaying on a screen said previously modified main video stream.

20. Electronic device including a computer memory storing instructions of a method according to claim 1.
